(19) United States
(12) Patent Application Publication
(10) Pub. No.: US 2024/0423493 A1
(43) Pub. Date: Dec. 26, 2024

(54) LUNG FLUID MONITOR AND MONITORING FLUID LEVEL IN A LUNG

(71) Applicant: Government of the United States of America, as represented by the Secretary of Commerce, Gaithersburg, MD (US)

(72) Inventor: Kamran Sayrafian-Pour, Rockville, MD (US)

(21) Appl. No.: 18/705,129

(22) PCT Filed: Oct. 28, 2022
(86) PCT No.: PCT/US2022/048217

§ 371 Date: Apr. 26, 2024

Related U.S. Application Data
(60) Provisional application No. 63/272,783, filed on Oct. 28, 2021.

(51) Int. Cl. A61B 5/05; A61B 5/00; A61B 5/08

(52) U.S. Cl. CPC A61B 5/05; A61B 5/08; A61B 5/4878; A61B 5/6804; A61B 5/7271; A61B 2560/0462; A61B 2562/043

(57) ABSTRACT

A lung fluid monitor monitors a fluid level in a lung and includes: a radiation source disposable on a first body surface and that produces nascent radiation, the nascent radiation: being received by the first body surface, communicated from the first body surface to a lung, attenuated proportionately to an amount of fluid in the lung, and communicated from the lung to a second body surface as attenuated radiation; a radiation detector disposable on the second body surface opposing the first body surface and in electromagnetic communication with the radiation source via the lung and that: receives the attenuated radiation from the lung and produces a detector signal from the attenuated radiation in response to receiving the attenuated radiation.

### CROSS REFERENCE TO RELATED APPLICATIONS

This application claims the benefit of U.S. Provisional Patent Application Ser. No. 63/272,783 (filed Oct. 28, 2021), which is herein incorporated by reference in its entirety.

### STATEMENT REGARDING FEDERALLY SPONSORED RESEARCH

This invention was made with United States Government support from the National Institute of Standards and Technology (NIST), an agency of the United States Department of Commerce. The Government has certain rights in this invention.

### BRIEF DESCRIPTION

Disclosed is a lung fluid monitor for monitoring fluid level in a lung, the lung fluid monitor comprising: a radiation source disposable on a first body surface and that produces nascent radiation, the nascent radiation: being received by the first body surface, communicated from the first body surface to a lung, attenuated proportionately to an amount of fluid in the lung, and communicated from the lung to a second body surface as attenuated radiation; a radiation detector disposable on the second body surface opposing the first body surface and in electromagnetic communication with the radiation source via the lung and that: receives the attenuated radiation from the lung and produces a detector signal from the attenuated radiation in response to receiving the attenuated radiation.

Disclosed is a process for monitoring fluid level in a lung with a lung fluid monitor, the process comprising: producing nascent radiation by a radiation source disposed on a first body surface; communicating the nascent radiation from the radiation source to a lung; receiving the nascent radiation by the lung; attenuating the nascent radiation in the lung to produce attenuated radiation from the nascent radiation; communicating the attenuated radiation from the lung to a radiation detector disposed on a second body surface that opposes the radiation source, such the lung is interposed between the first body surface and the second body surface; receiving, by the radiation detector, the attenuated radiation from the lung; producing, by the radiation detector, a detector signal from the attenuated radiation; communicating the detector signal from the radiation detector to a analyzer; receiving, by the analyzer, the detector signal from the radiation detector; analyzing, by the analyzer, the detector signal; producing, by the analyzer, lung health data from analyzing the detector signal to monitor the fluid level in a lung.

### BRIEF DESCRIPTION OF THE DRAWINGS

The following description cannot be considered limiting in any way. Various objectives, features, and advantages of the disclosed subject matter can be more fully appreciated with reference to the following detailed description of the disclosed subject matter when considered in connection with the following drawings, in which like reference numerals identify like elements.

FIG. 1 shows a patient, according to some embodiments.

FIG. 2 shows a lung fluid monitor disposed on a patient, according to some embodiments.

FIG. 3 shows a lung fluid monitor, according to some embodiments.

FIG. 4 shows a lung fluid monitor, according to some embodiments.

FIG. 5 shows a lung fluid monitor, according to some embodiments.

FIG. 6 shows a lung fluid monitor, according to some embodiments.

FIG. 7 shows a lung fluid monitor, according to some embodiments.

FIG. 8 shows: (A) a lung fluid monitor and (B) a plurality of waveforms for non-overlapping pulses of nascent radiation from different radiation sources, according to some embodiments.

FIG. 9 shows a lung fluid monitor with discernment of attenuated radiation occurring in various lung regions, according to some embodiments.

FIG. 10 shows a shirt with a lung fluid monitor embedded in the shirt, according to some embodiments.

FIG. 11 shows a graph of attenuated radiation intensity versus mass density for analyzed data acquired from lung fluid monitor, according to some embodiments.

FIG. 12 shows a computational model of lungs that includes a lattice of macro-alveolus, according to some embodiments.

FIG. 13 shows a computational model of a lung that includes a lattice of macro-alveolus representing fluid accumulation in a bottom or top half of lung, according to some embodiments.

FIG. 14 shows a computational model of a human body and positions of antennas on each lung (N=2), according to some embodiments.

FIG. 15 shows a rectangular loop antenna in (a) isometric view and (b) front view, according to some embodiments.

FIG. 16 shows a graph of reflection coefficient (S11) of the antenna for various mass densities, according to some embodiments.

FIG. 17 shows a deviation matrix D^{ρ} when the right lung experiences excess fluid, according to some embodiments.

FIG. 18 shows a deviation matrix D^{ρ} when the left lung experiences excess fluid, according to some embodiments.

FIG. 19 shows a graph for functions R(ρ) and L(ρ), according to some embodiments.

FIG. 20 shows deviations in a sum of magnitudes of a detector signal when a right lung has excess fluid (N=2), according to some embodiments.

FIG. 21 shows deviations in a sum of magnitudes of a detector signal when a left lung has excess fluid (N=2), according to some embodiments.

FIG. 22 shows an ∠S₂₁^{ρ} (1, 1) with (a) increasing excess fluid in the top half of the right lung (b) no excess fluid in the top half but increasing excess fluid in the bottom half of the right lung, according to some embodiments.

FIG. 23 shows an ∠S₂₁^{ρ} (3,3) with (a) no excess fluid in the bottom half but increasing excess fluid in the top half of the right lung (b) increasing excess fluid in the bottom half of the right lung, according to some embodiments.

### DETAILED DESCRIPTION

A detailed description of one or more embodiments is presented herein by way of exemplification and not limitation.

Pulmonary edema is a medical condition caused by the accumulation of fluids in the lungs. Diagnosing the severity of this fluid buildup typically requires the use of expensive medical imaging systems. For patients who are far away from a hospital and who might require continuous monitoring this could be a problem. The lung fluid monitor includes an antenna that can be placed on the patient's chest and back area. Guided by computational models, changes in the RF signal transmitted through the body correlates with the amount of fluid in the lungs. Hence, continuous measurement of this RF signal through a wearable band or a T-shirt with embedded electronics could be an inexpensive monitoring system to detect the status of pulmonary edema.

It has been discovered that lung fluid monitor 200 provides a wireless, wearable system that monitors a level of fluid in the lungs. Operation of lung fluid monitor 200 can be implemented as a personal device for monitoring buildup of fluid in the lungs and can decrease trips to hospitals for remote patients or when hospital resources are scarce. Conventional devices cannot be used at home, but lung fluid monitor 200 can be used at one's home.

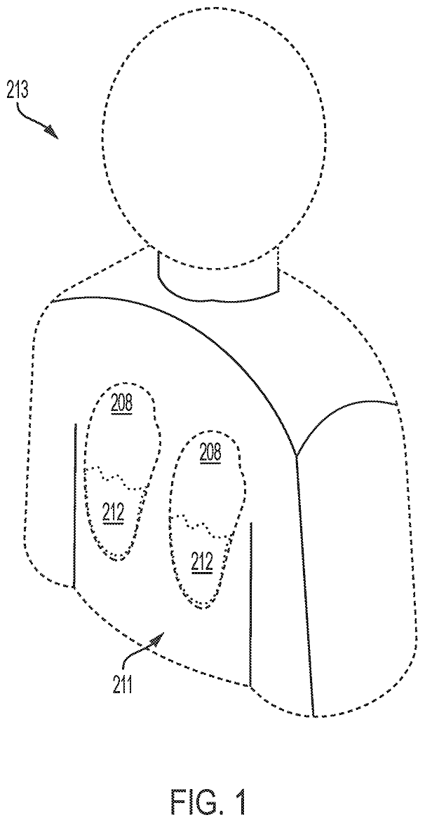

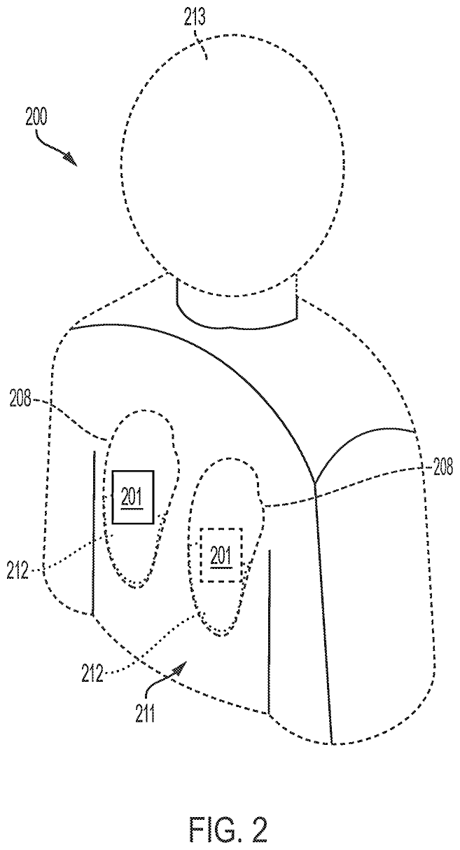

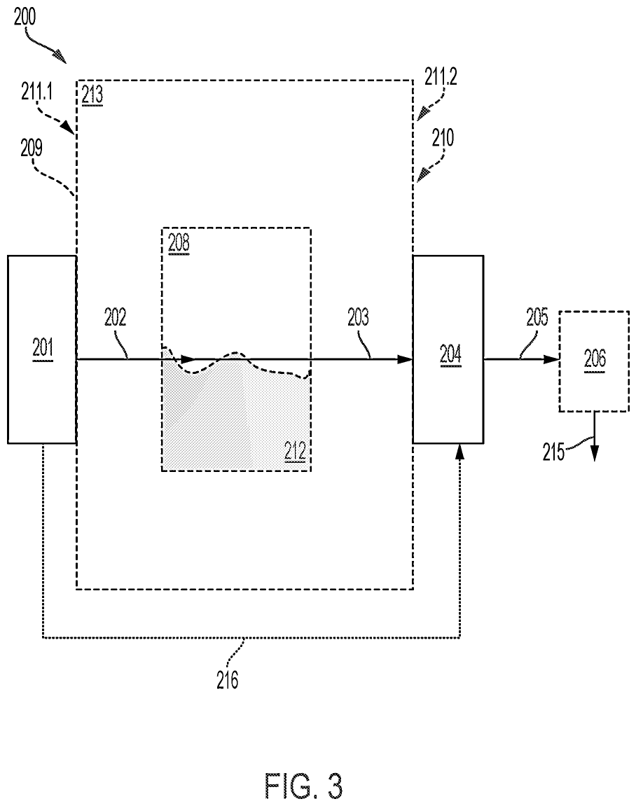

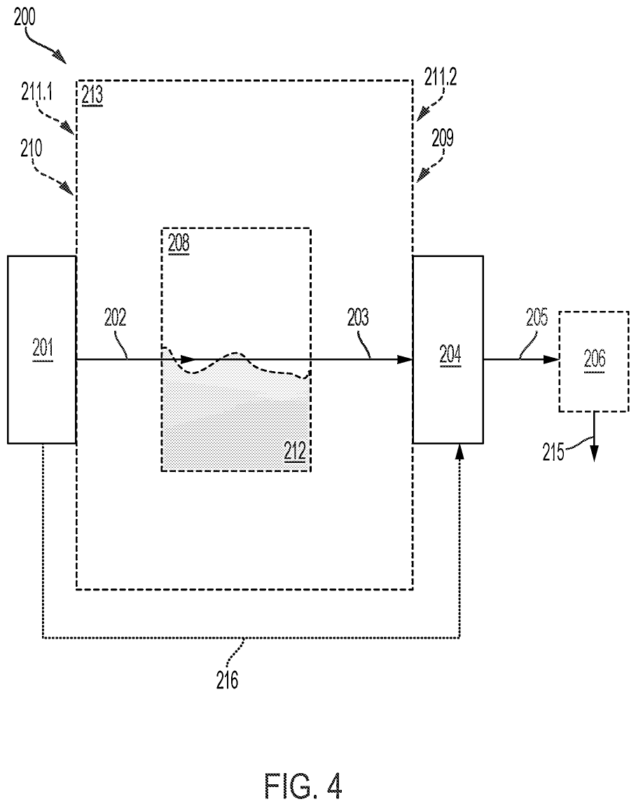

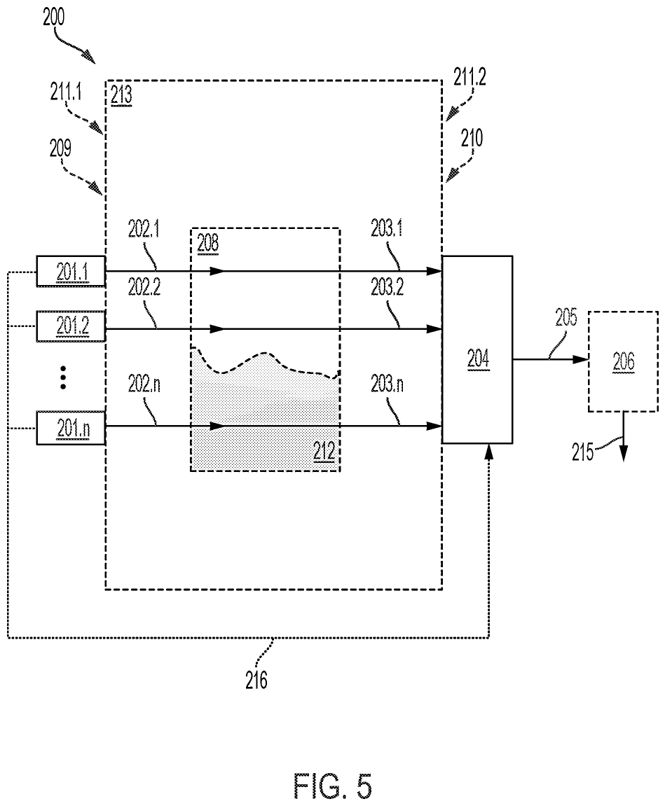

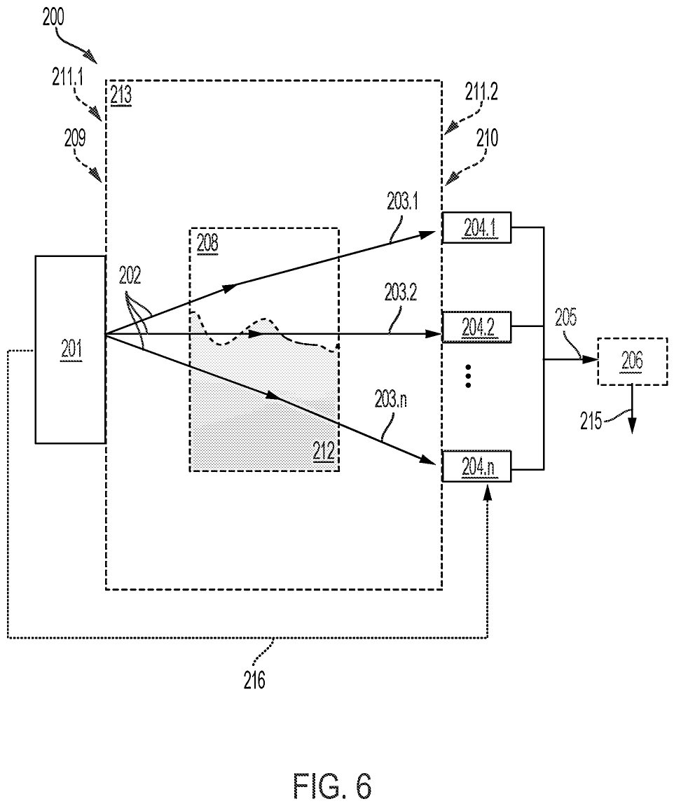

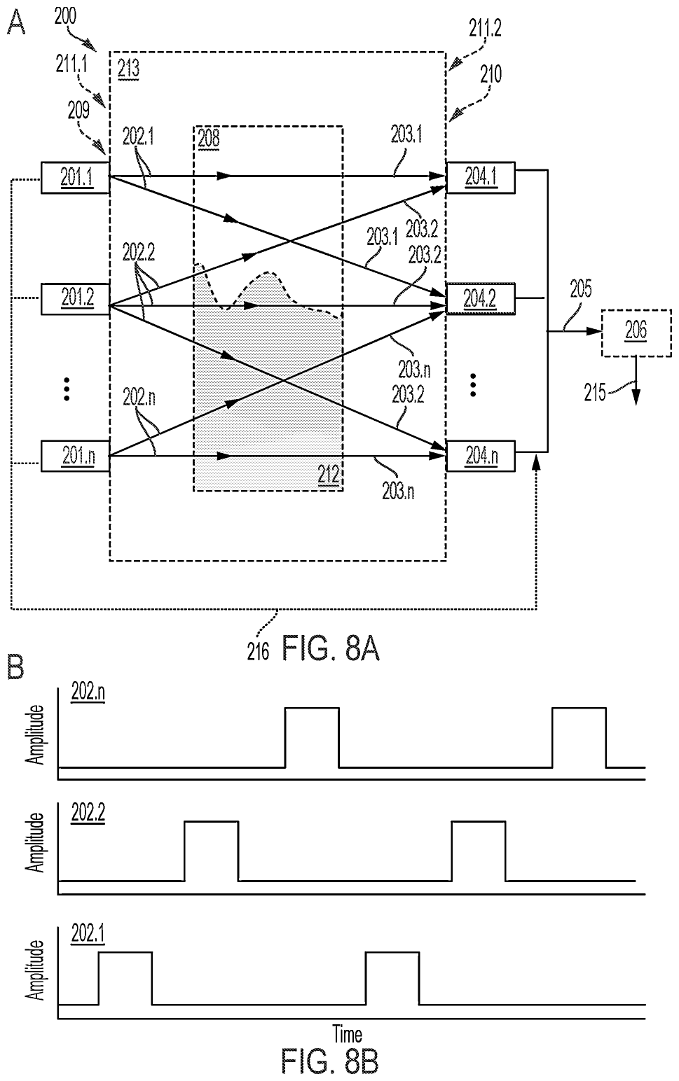

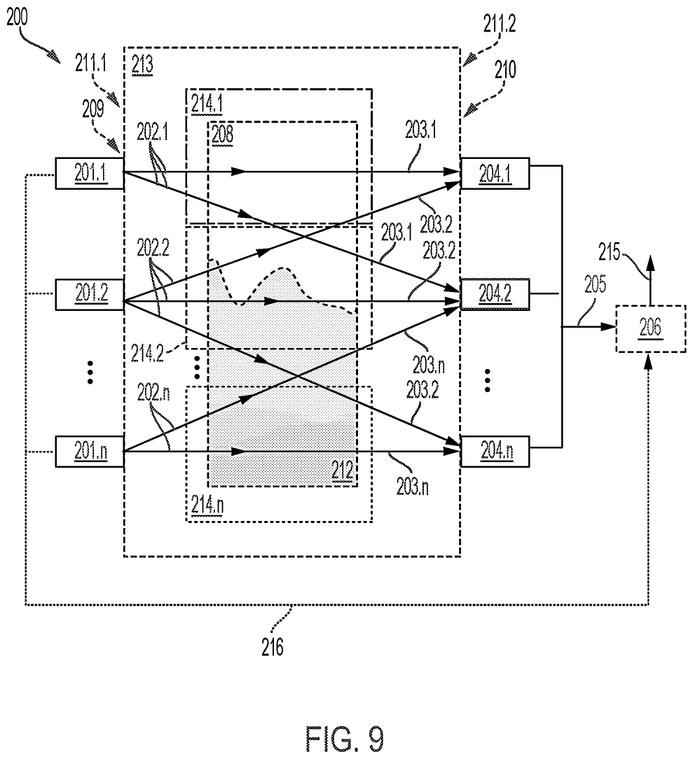

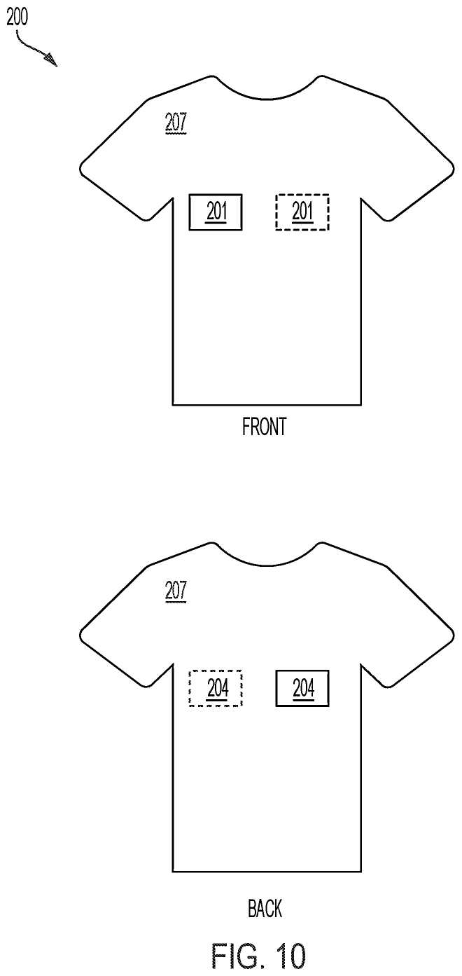

Lung fluid monitor 200 performs monitoring fluid level in a lung. In an embodiment, with reference to FIG. 1, FIG. 2, FIG. 3, FIG. 4, FIG. 5, FIG. 6, FIG. 8, FIG. 9, and FIG. 10, lung fluid monitor 200 includes: a radiation source 201 disposable on a first body surface 211.1 and that produces nascent radiation 202, the nascent radiation 202: being received by the first body surface 211.1, communicated from the first body surface 211.1 to a lung 208, attenuated proportionately to an amount of fluid 212 in the lung 208, and communicated from the lung 208 to a second body surface 211.2 as attenuated radiation 203; a radiation detector 204 disposable on the second body surface 211.2 opposing the first body surface 211.2 and in electromagnetic communication with the radiation source 201 via the lung 208 and that: receives the attenuated radiation 203 from the lung 208 and produces a detector signal 205 from the attenuated radiation 203 in response to receiving the attenuated radiation 203.

In an embodiment, lung fluid monitor 200 includes analyzer 206 in electrical communication with the radiation detector 204 and that receives the detector signal 205 from the radiation detector 204 and produces lung health data 215 from the detector signal 205. In an embodiment, radiation source 201 produces a phase signal 216 that is communicated from the radiation source 201 and received by the analyzer 206. In an embodiment, radiation source 201 comprises a signal generator that produces a waveform for the nascent radiation 202. In an embodiment, radiation source 201 further comprises a source antenna that receives the source waveform from the signal generator, produces the nascent radiation 202 from the source waveform, and communicates the nascent radiation 202 to the first body surface 211.1. In an embodiment, radiation detector 204 comprises a detection antenna that receives the attenuated radiation 203 from the lung 208. In an embodiment, radiation detector 204 further comprises a data processor that converts the attenuated radiation 203 into the detector signal 205. In an embodiment, radiation detector 204 further comprises a transmitter that communicates the detector signal 205 to the analyzer 206.

In an embodiment, with reference to FIG. 10, lung fluid monitor 200 includes housing member 207 on which the radiation source 201 and the radiation detector 204 are disposed. In an embodiment, housing member 207 is wearable garment that is worn by a human. Exemplary wearable garments include a shirt, vest, coat, undergarment (e.g., undershirt, brassiere, and the like), and the like. According to an embodiment, housing member 207 is a shirt or vest with embedded electronics that operate radiation source 201 and radiation detector 204.

In an embodiment, wherein the nascent radiation 202 and the attenuated radiation 203 independently comprise a frequency compliant with the United States of America Federal Communications Communication MedRadio spectrum. In an embodiment, the MedRadio spectrum is from 401 MHz to 457 MHz. The MedRadio spectrum is specified at Title 47 of the United States of America Code of Federal Regulations § 95.2563 (last amended Oct. 24, 2022) for MedRadio frequency bands. Medical Device Radiocommunications Service (MedRadio) can be apportioned from 401 MHz to 406 MHZ, 413 MHz to 419 MHz, 426 MHz to 432 MHz, 438 MHz to 444 MHZ, or 451 MHz to 457 MHz. It should be appreciated that the MedRadio spectrum is used for diagnostic and therapeutic purposes in implanted medical devices as well as devices worn on a body, such as lung fluid monitor 200. Other MedRadio devices include implanted cardiac pacemakers and defibrillators as well as neuromuscular stimulators that help restore sensation, mobility, and other functions to limbs and organs. Medical Body Area Networks (MBANs), which are low power networks of sensors worn on the body controlled by a hub device that is located either on the body or in close proximity to it, operate in the 2360-2400 MHz band.

Lung fluid monitor 200 can be made of various elements and components that are fabricated or assembled from components. Elements of lung fluid monitor 200 can be various sizes that can be made of a material that is physically or chemically resilient in an environment in which lung fluid monitor 200 is disposed. Exemplary materials include a metal, ceramic, thermoplastic, glass, semiconductor, and the like. The elements of lung fluid monitor 200 can be made of the same or different material and can be monolithic in a single physical body or can be separate members that are physically joined.

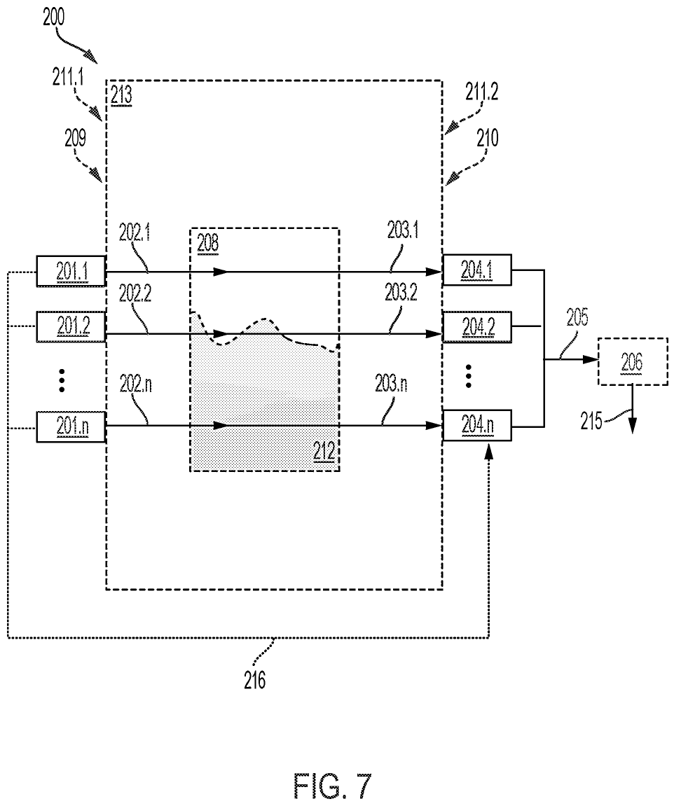

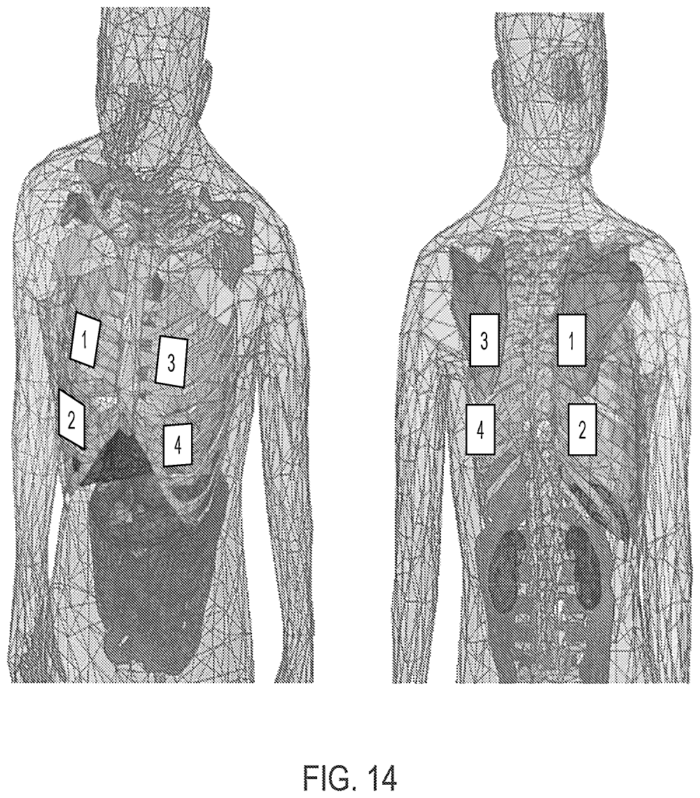

The number and arrangement of radiation source 201 and radiation detector 204 is arbitrary. In an embodiment, with reference to FIG. 5, FIG. 7, FIG. 8, FIG. 9, FIG. 10, and FIG. 14, lung fluid monitor 200 includes additional radiation sources 201 disposed in an array for monitoring the lung 208. In an embodiment, with reference to FIG. 6, FIG. 7, FIG. 8, FIG. 9, FIG. 10, and FIG. 14, lung fluid monitor 200 includes additional radiation detectors 204 disposed in an array for monitoring the lung 208. In an embodiment, with reference to FIG. 5, FIG. 6, FIG. 7, FIG. 8, FIG. 9, FIG. 10, and FIG. 14, lung fluid monitor 200 includes additional radiation sources 201 disposed in an array; and additional radiation detectors 204 disposed in an array for monitoring the lung 208, wherein the additional radiation sources 201 are in electromagnetic communication with the additional radiation detectors 204.

Lung fluid monitor 200 can include radiation sources 201 to cover different lobes (or areas) of the left and right lungs 208. Multiple radiation detectors 204 can be disposed on patient 213. A set of N radiation sources 201 and N radiation detectors 204 provides N×N electromagnetic communication links, and N×N detector signals in detector signal 205. The set of these N×N detector signals provide information on a location of fluid accumulation or position of fluid in lungs 208.

It should be appreciated that one or both lungs can be monitored by lung fluid monitor 200 independently of one another. Further, operation of individual radiation sources 201 can be adjusted so that, when a plurality of radiation source 201 (e.g., 201.1, 201.2, . . . , 201.n, wherein n is an arbitrary selected number of radiation sources 201) are operated, nascent radiation 202 (e.g., 202.1 from 201.1, 202.2 from 201.2, . . . , 202.n from 202.n) from different radiation sources 201 have a selectively tailored waveform as shown in FIG. 8B. Here, pulses of nascent radiation 202 from different radiation sources 201 are not temporally overlapping. As a result, the various attenuated radiation 203 (e.g., attenuated radiation 203.1, attenuated radiation 203.2, . . . , attenuated radiation 203.n) received at radiation detector 204 can be associated with the particular radiation source 201 that produced the specific attenuated radiation 203 received by radiation detector 204 because each attenuated radiation 203, although attenuated with respect to the amplitude of nascent radiation 202, includes the temporal aspects of the waveform, and the zero of time associated with production of the plurality of waveforms for all of nascent radiation 202.i (i=1, 2, . . . , n) waveforms can be synchronized across all radiation sources 201. Moreover, as shown in FIG. 9 various radiation sources 201 and radiation detectors 204 samples certain lung regions 214 so that detector signal 205 includes location information of particular lung regions 214 associated with fluid in specific parts of lung 208.

With regard to disposition of radiation source 201 and radiation detector 204 on patient 213, radiation source 201 and radiation detector 204 oppose one another on opposing body surface 211. It is contemplated that radiation source 201 can be disposed on ventral surface 209, while radiation detector 204 is disposed on dorsal surface 210. In some embodiments, radiation source 201 is disposed on dorsal surface 210, and radiation detector 204 is disposed on ventral surface 209.

In an embodiment, radiation source 201 includes electronic components to produce and communicate nascent radiation 202. Radiation source 201 can includes a power source (e.g., a battery), programmable electronics to set the timing and frequency of measurements as well as a counter to account for the number of sources, a waveform generator at a desired frequency and magnitude within the MedRadio spectrum plus additional on/off circuitry to start and stop the waveform, power amplifier, and wearable antennas. Radiation source 201 can include electronics to shape and steer the radiation pattern generated by wearable antennas. The steerable radiation pattern of nascent radiation 202 produced by radiation source 201 can scan lung 208 or focus nascent radiation 202 in a specific direction for monitoring purposes. Radiation source 201 (or radiation detector 204) can include electronics to control the timing and frequency of the measurements. Timing of the measurement can be controllable to synchronize with fully inhaled, fully exhaled or continuous measurement during inhalation process. A frequency of acquiring measurements can be minutes, hours, or daily.

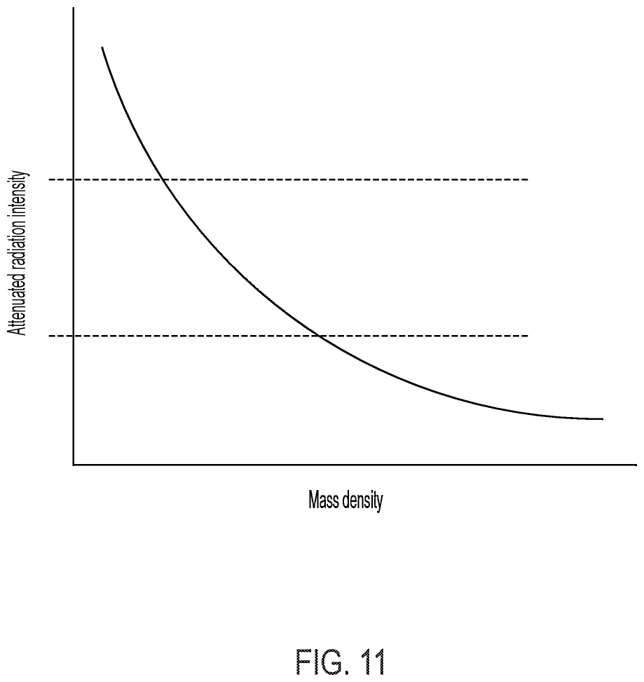

In an embodiment, radiation detector 204 includes electronic components to receive attenuated radiation 203 and produce and communicate detector signal 205. Radiation detector 204 can include a wearable antennas, power amplifiers, and waveform filters. Detector signal (or signals) 205 include electrical components for attenuated radiation 203 such as intensity. This radiation intensity is a decreasing function of the mass density of the lung (FIG. 11). Since mass density of the lung is proportional to the volume of the fluid in the lungs, any change in the radiation intensity (measured at the same instant of lung inhalation) will indicate a change in the lung fluid. Monitoring the fluid level change in the human lung occurs by monitoring the radiation intensity of signals 205.

Analyzer 206 includes electronics to measure magnitude of the received waveform, electronics to measure the phase difference of the received waveform with the radiated waveform, electronics to store and perform computations on the sequence of magnitudes and phase differences along with the measurement time and date. Analyzer 206 can include electronics to connect and transmit information to other devices such as smart phones, laptops, or Wi-Fi routers, e.g., at home. Analyzer 206 can include electronics to connect to a mobile phone or Internet, to remotely monitor fluid levels, to remotely control lung fluid monitor 200, or to store measurement data on a patient's phone or a server at a healthcare facility.

Lung fluid monitor 200 can be made in various ways. It should be appreciated that lung fluid monitor 200 includes a number of optical, electrical, or mechanical components, wherein such components can be interconnected and placed in communication (e.g., optical communication, electrical communication, mechanical communication, and the like) by physical, chemical, optical, or free-space interconnects. As a result, lung fluid monitor 200 can be disposed in a terrestrial environment or space environment. Elements of lung fluid monitor 200 can be formed from suitable materials that can include textiles, ceramic, glass, or metal. According to an embodiment, the elements of lung fluid monitor 200 are formed using 3D printing although the elements of lung fluid monitor 200 can be formed using other methods, such as injection molding or machining a stock material such as block of material that is subjected to removal of material such as by cutting, laser oblation, and the like. Accordingly, lung fluid monitor 200 can be made by additive or subtractive manufacturing.

Lung fluid monitor 200 has numerous advantageous and unexpected benefits and uses. In an embodiment, a process for monitoring fluid level in a lung includes: producing nascent radiation 202 by a radiation source 201 disposed on a first body surface 211.1; communicating the nascent radiation 202 from the radiation source 201 to a lung 208; receiving the nascent radiation 202 by the lung 208; attenuating the nascent radiation 202 in the lung 208 to produce attenuated radiation 203 from the nascent radiation 202; communicating the attenuated radiation 203 from the lung 208 to a radiation detector 204 disposed on a second body surface 211.2 that opposes the radiation source 201, such the lung 208 is interposed between the first body surface 211.1 and the second body surface 211.2; receiving, by the radiation detector 204, the attenuated radiation 203 from the lung 208; producing, by the radiation detector 204, a detector signal 205 from the attenuated radiation 203; communicating the detector signal 205 from the radiation detector 204 to a analyzer 206; receiving, by the analyzer 206, the detector signal 205 from the radiation detector 204; analyzing, by the analyzer 206, the detector signal 205; producing, by the analyzer 206, lung health data 215 from analyzing the detector signal 205 to monitor the fluid level in a lung.

In an embodiment, monitoring fluid level in a lung includes communicating a phase signal 216 from the radiation source 201 to the analyzer 206. In an embodiment, monitoring fluid level in a lung includes determining a lung region 214 of the lung 208 corresponding to the lung health data 215.

In an embodiment, lung health data 215 includes a regional fluid accumulation in a lung region 214. In an embodiment, monitoring fluid level in a lung includes a temporal history a change in an amount of fluid in the lung 208.

The absolute value of the fluid level in lung 208 depends on the lung size or condition that can vary across different people. Lung fluid monitor 200 measures increase or decrease of fluid in lung 208. Consecutive or periodic measurements are involved to compare relative radio frequency readings with previous readings to determine whether fluid amount is increasing, decreasing, or the same.

Lung fluid monitor 200 performs measurement periodically. A period of measurements can be adjusted to minutes, hours, or days. If radiation sources (201.1, . . . , 201.n) or radiation detectors (204.1, . . . , 204.n) do not include electronics to create a steerable radiation pattern, the measurement cycle includes various operations. Radiation sources 201 on the right lung transmit nascent radiation 202, e.g., within the MedRadio frequency band, with a constant intensity. The signal transmissions are consecutive and without overlap from radiation sources (201.1, . . . , 201.n) in order. Then radiation sources (201.1, . . . , 201.n) disposed to cover the left lung continue orderly transmission in a similar manner. The timing between consecutive transmissions can be adjustable. Radiation detectors (204.1, . . . , 204.n) on the right lung as well as radiation detectors (204.1, . . . , 204.n) on the left lung simultaneously receive the consecutive transmissions of attenuated radiation 203 and produce and communicate detector signal 205 to analyzer 206.

After receiving detector signal 205, analyzer 206 performs several analysis operations. The sum of the magnitudes of the detector signal 205 for each transmission of nascent radiation 202 from radiation sources (201.1, . . . , 201.n) located on the right lung is calculated. The value of this sum is compared with the previous cycle result. A higher value for this sum indicates that the excess fluid in the right lung is decreasing. A lower value indicates that the excess fluid in the right lung is increasing.

The sum of the magnitudes of the detector signal 205 for each transmission of nascent radiation 202 from radiation sources (201.1, . . . , 201.n) located on the left lung is calculated. The value of this sum is compared with the previous cycle result. A higher value for this sum indicates that the excess fluid in the left lung is decreasing; while a lower value indicates that the excess fluid in the left lung is increasing.

For the right lung, the phase difference between the radiation source 201.1 and radiation detector 204.1 is calculated, wherein phase signal 216 is received by analyzer 206 as shown, e.g., in FIG. 3. The value of this phase difference is compared with the previous cycle result. A higher phase difference indicates that the excess fluid in the region of the right lung located directly between radiation source 201.1 and radiation detector 204.1 is increasing. A lower phase difference indicates that the excess fluid in that region is decreasing. This procedure can be repeated for radiation source/detector pairs 201.2/204.2 to radiation source/detector 201.n/204.n. The change in the phase difference between each pair similarly indicates the status of the excess fluid at each region of the right lung located directly between the corresponding radiation source/detector pair.

For the left lung, the phase difference between the radiation source 201.1 and radiation detector 204.1 is calculated. The value of this phase difference is compared with the previous cycle result. A higher phase difference indicates that the excess fluid in the region of the left lung located directly between radiation source 201.1 and radiation detector 204.1 is increasing. A lower phase difference indicates that the excess fluid in that region is decreasing. This process is repeated for radiation source/detector pairs 201.2/204.2 to radiation source/detector 201.n/204.n. The change in the phase difference between each pair similarly indicates the status of the excess fluid at each region of the left lung located directly between the corresponding radiation source/detector pair.

If the radiation sources 201.1 to 201.n or radiation detectors 204.1 to 204.n include electronics to create a steerable radiation pattern to scan each lung or focus the electromagnetic radiation in a specific direction for monitoring purposes, then each measurement cycle in the lung fluid monitor 200 involves the various steps. The radiation sources 201.1 to 201.n on the right lung produce a radiation pattern of nascent radiation 202 focused on radiation detector 204.1 and transmit a signal within the MedRadio frequency band with a constant intensity. The radiation detector 204.1 on the right lung receives the transmitted signal and passes it on to the analyzer via the detector signal 205. The radiation sources will then steer the radiation pattern toward radiation detectors 204.2 to 204.n and repeat signal transmission and reception by each detector in order. The timing between consecutive transmissions can be adjustable.

The radiation sources 201.1 to 201.n on the left lung will produce a radiation pattern of nascent radiation 202 focused on radiation detector 204.1 and transmits a signal within the MedRadio frequency band with a constant intensity/magnitude. The radiation detectors 204.1 on the left lung receives the transmitted signal and passes it on to the analyzer via the detector signal 205. The radiation sources will then steer the radiation pattern toward radiation detectors 204.2 to 204.n and repeat signal transmission and reception by each detector in order. The timing between consecutive transmissions can be adjustable.

After receiving the detector signal 205, the analyzer 206 performs various analysis operations. The sum of the magnitudes of the detector signal 205 for each transmission from the steerable radiation pattern created by sources 201.1 to 201.n located on the right lung is calculated. The value of this sum is compared with the previous cycle result. A higher value for this sum indicates that the excess fluid in the right lung is decreasing; while a lower value indicates that the excess fluid in the right lung is increasing.

The sum of the magnitudes of the detector signal 205 for each transmission from the steerable radiation pattern created by sources 201.1 to 201.n located on the left lung is calculated. The value of this sum is compared with the previous cycle result. A higher value for this sum indicates that the excess fluid in the left lung is decreasing. A lower value indicates that the excess fluid in the left lung is increasing.

For the right lung, the phase difference between the transmitted signal from the radiation sources 201.1 to 201.n when the radiation pattern is focused on detector 204.1 and signal received at the radiation detector 204.1 is calculated. The value of this phase difference is compared with the previous cycle result. A higher phase difference indicates that the excess fluid in the region of the right lung covered by the focused radiation pattern which is directed toward detector 204.1 is increasing. A lower phase difference indicates that the excess fluid in that region is decreasing. This process is repeated when the radiation pattern steers towards detectors 201.2 to 204.n. The change in the phase difference as the radiation pattern focuses on each detector similarly indicates the status of the excess fluid at each corresponding region of the right lung.

For the left lung, the phase difference between the transmitted signal from the radiation sources 201.1 to 201.n when the radiation pattern is focused on detector 204.1 and signal received at the radiation detector 204.1 is calculated. The value of this phase difference is compared with the previous cycle result. A higher phase difference indicates that the excess fluid in the region of the left lung covered by the focused radiation pattern which is directed toward detector 204.1 is increasing. A lower phase difference indicates that the excess fluid in that region is decreasing. This process is repeated when the radiation pattern steers towards detectors 201.2 to 204.n. The change in the phase difference as the radiation pattern focuses on each detector similarly indicates the status of the excess fluid at each corresponding region of the left lung.

The articles and processes herein are illustrated further by the following Example, which is non-limiting.

### Example

Excess fluid in the human lungs is a medical condition referred to as pulmonary edema. Here, a wearable system that can be used at home is described to detect or monitor fluid levels in the lungs. The wearable system includes wearable antennas operating at the MedRadio frequency band and optimized for signal penetration through the body. The flexible antennas can be integrated in a wearable device and placed on the chest and back of a human to adequately have monitoring coverage of the lungs. Using the channel responses between selected pairs of antennas, the device monitors the level of fluid accumulation in the lung. The device identifies the region of the lung where fluid is concentrated.

Simulations using a 3D human body model with a computational model of the lung emulating various levels of fluid were conducted. The simulation results confirm that the device is a wearable technology for remote or self-monitoring of the lungs. The device can be used by both patients who are susceptible to pulmonary edema and those who are recovering still under continuous observation.

The accumulation of excess fluid in the human lungs is a medical condition referred to as pulmonary edema. This is a common condition in elderly with about 1 in 15 people aged 75-84 and just over 1 in 7 people aged 85 years and above. Heart problem, pneumonia, exposure to certain toxins, as well as viruses such as COVID-19 could be the cause for such excess fluid. If this condition is not treated in time, shortness of breath sets in, leading to acute respiratory distress syndrome (ARDS) which is a form of lung failure. X-ray, CT scan, and Electrical Impedance Tomography are typical technologies that are used to detect and measure fluid build-up in the lungs; however, physical presence and access of the patient to healthcare facilities having relevant equipment are required for accurate diagnosis. For patients living in remote areas or people who need to be continuously monitored, frequent access to such facilities might not be easily feasible or convenient. In addition, during pandemics, resources at medical facilities are typically diverted to other critical tasks. Therefore, it is desirable to cut down the number of unnecessary visits by the patients during those times.

Applications of the Internet-of-Things (IoT) technology to health monitoring and telemedicine have received considerable attention since the COVID-19 pandemic. Extension of health monitoring services to people's home through personal wearable devices not only reduces the cost of healthcare services but could also ensures higher quality of life for some patients. Proliferation and consumer adoption of wearable devices have also created a fertile environment for new applications that enable health monitoring functions to take place at patients' home.

The Medical Device Radiocommunications Service (MedRadio) consists of several frequency bands in the low to mid-400 MHZ range. MedRadio spectrum is used for diagnostic and therapeutic purposes in implanted as well as wearable devices. The 401-406 MHz band is the lowest frequency band for MedRadio devices. Although designing small wearable antennas at low MedRadio frequencies is a challenge, lower frequencies offer better penetration through the lossy environment of the human body. At these frequencies, and compared to a normal lung tissue, the composition of the lungs with excess fluid (such as saline) would provide a different effective dielectric media (i.e., conductivity and permittivity) for a wireless signal passing through the lungs. Change in the environment due to excess fluid effectively impacts the propagation channel, and consequently the time and frequency responses of the channel or equivalently the corresponding scattering parameters.

By observing this change in the propagation environment, the wearable device can be used to detect fluid buildup in human lungs. The device can include a pair of wearable antennas (optimized for radio wave transmission through the body at MedRadio frequency band) to indicate a correlation between the communication channel response and the level of fluids inside the lungs can be exploited to monitor the increase (or decrease) of the amount of excess fluid. In some of these devices, multiple antennas are placed on the front and back side of the chest to 1) increase the sensitivity of fluid level monitoring and 2) allow the detection of the region in a lung where fluid buildup is concentrated. Using the computational model of a lung representing excess fluid, an extensive set of simulations are conducted with the 3D human body area networking platform at the Information Technology Laboratory of the National Institute of Standards and Technology (NIST). The simulation results clearly confirm that the system monitors variations in the fluid levels of the lungs (with high sensitivity) as well as specific region of the lung (i.e., top versus bottom half) where excess fluid is concentrated.

### Computational Modeling of the Lung

The tiny air sacs (known as alveoli) at the very end of the bronchial tree (i.e., air tubes branches in the lungs including bronchioles, and bronchi) are responsible for the oxygen exchange with carbon dioxide during the breathing process. In pulmonary edema, the alveoli are partially filled with fluid; interfering with the normal patient's breathing process. On average, there are 480 million of alveoli in the human lungs, and the mean size of a single alveolus is estimated to be around 4.2×10⁶ μm³. Computational modeling of such small structures within the lung tissue is nearly impossible. However, such microscopic level of modeling might not be necessary for the purpose of this study. The accumulating effect of fluid buildup in the alveoli is the change in the dielectric properties of the lung's tissue (i.e., the medium where the electromagnetic waves travel through). However, as knowledge of these dielectric properties for various levels of fluid is not currently available, we propose a macroscopic level of computational modeling where a cluster of alveoli are considered as one macro-alveolus. As such, the following methodology is used to construct a lung model incorporating the effect of fluid buildup. A periodic lattice of small elements (e.g., cubes) is created inside the volume of the lungs. Each cube (hereafter referred to as Lattice Element (LE)) represents the overall volume of fluid that has been accumulated in a macro-alveolus. To remove the periodicity of the lattice, an algorithm has been developed to randomize the spacing between the LEs. It should be noted that since the dimensions of the lattice elements is much smaller than the wavelength of the RF signal that is used in this study, the actual shape of the lattice elements (i.e., cubes) will not have any impact on the final results and other shapes such as spheres can be used.

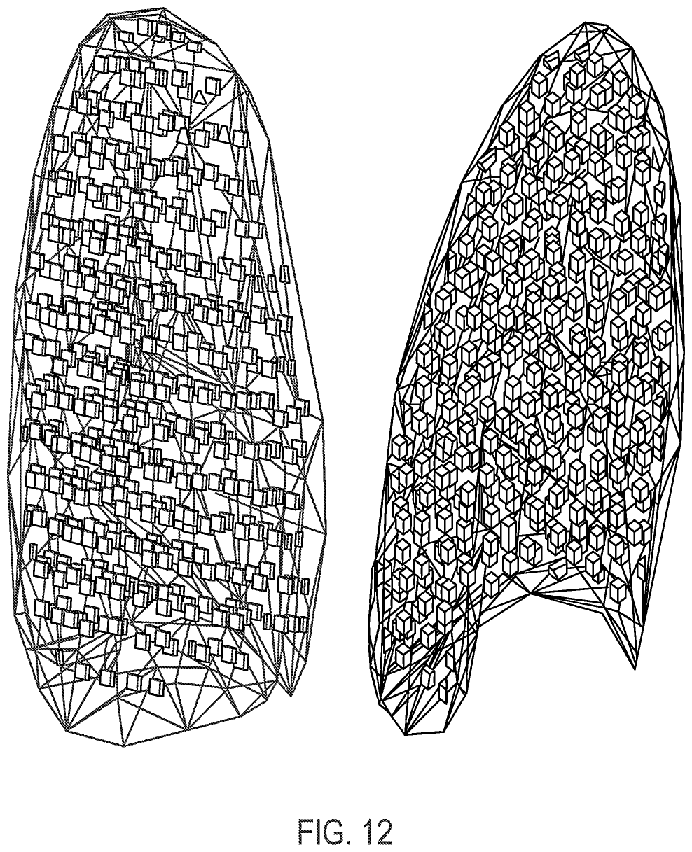

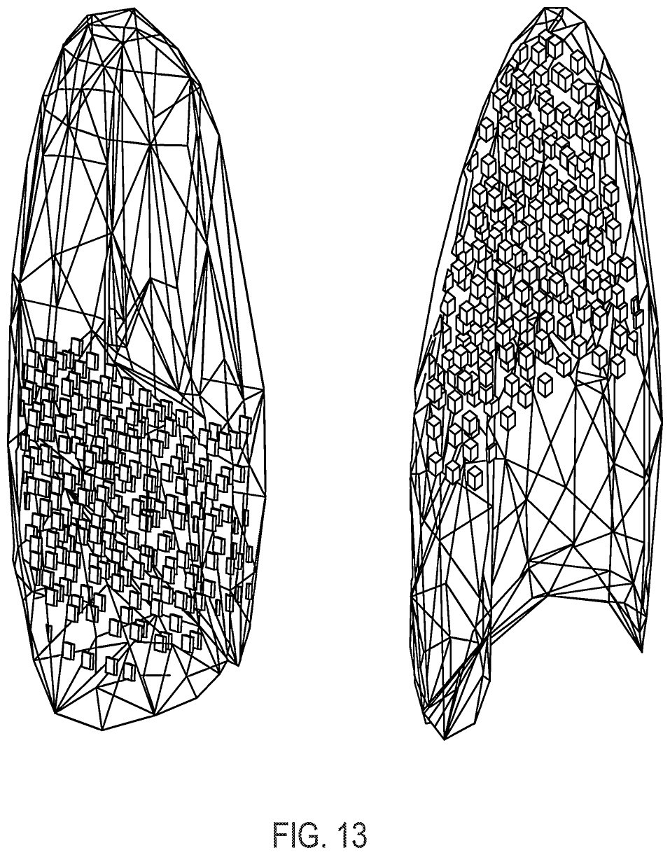

FIG. 12 shows a sample of the lung model including the randomized lattice structure covering the whole lung. Similarly, FIG. 13 shows the lung model with the randomized macro-alveolus lattice covering the bottom or top half of the lung. The two parameters in this lung model are the LE size and the average spacing between two adjacent LEs. The average spacing will determine the total number of LEs that are contained within the volume of the lungs. The number of LEs along with their size will also determine the total volume of the LEs that exist inside the lungs. This volume indicates the total fluid buildup which impacts the overall mass density of the lungs representing pulmonary edema. This mass density can be calculated according to the following formula:

$${ρ}_{lung}^{( LE size , LE number )} = \frac{{ρ}_{lung}^{( 0 , 0 )} \times ( {V}_{lung} - {V}_{fluid}^{( LE size , LE number )} ) + {ρ}_{fluid} \times {V}_{fluid}^{( LE size , LE number )}}{{V}_{lung}}$$

where

ρ_{lung}^{(LE size,LE number)}=Mass density of the lungs with excess fluid

ρ_{lung}^{(0,0)}=Mass density of the normal lungs tissue(fully inhaled)

ρ_{fluid}=Mass density of the fluid inside the lungs

V_{fluid}^{(LE size,LE number)}=Total volume of the fluid inside the lungs fluid

V_{lung}=Total volume of the lungs.

The substance of the fluid that builds up inside the alveoli is assumed to be 0.9% saline solution. Therefore, ρ_{fluid}=ρₛₐₗᵢₙₑ=1.0046 g/cm³. Electrical properties of lossy liquids such as sodium chloride (NaCl) are very much temperature dependent. Using the polynomial equations in, we can calculate conductivity and permittivity of sodium chloride solutions as a function of normality and temperature. The temperature range where the model in was validated with actual data was 5-35° C. However, assuming that the model is still valid for 37° C. (i.e., the normal human body temperature) and using a concentration/molarity of 0.154 mol/L for sodium chloride solutions, the dielectric properties of 0.9% saline solution at body temperature (37° C.) can be estimated to be 72.47 F/m in permittivity and 1.86 S/m in conductivity.

The average mass density of the normal (fully inhaled) lung tissue (ρₜᵢₛₛᵤₑ) is known to be 0.2 g/cm³. Assuming a dimension of 6 mm for an individual cubic-shaped LE (equivalent to a volume of 216×10⁹ μm³) and considering that the volume of the 3D computational lung model in our simulation is V_{lung}=2309760.59 mm³, Table 1 shows the resulting mass densities of the lungs for various concentration and number of macro-alveoli spread across the whole volume of the lung. Similarly, Table 2 shows the same variations for the case when macro-alveoli are concentrated only in the top or bottom half of the lung volume. This mass density is used as a parameter to study the impact of fluid accumulation on the forward transmission coefficient of the wireless channels between multiple pairs of wearable antennas that will be discussed in the next section. The possible range of this mass density parameter is from 0.2 g/cm³ (indicating healthy lung tissue, fully inhaled) to an extreme 1.0 g/cm³ where the whole lung volume is basically considered to be filled with 0.9% saline solution. Table 1 only shows the relevant information up to 0.44 g/cm³ for full lung and 0.32 g/cm³ for half lung.

The detection of variations in mass densities in the lower range (i.e., up to 0.44 g/cm³) can be a focus for home or remote monitoring applications. Beyond that the patient can transfer to a hospital or clinic to receive care.

TABLE 1
Lung mass density variation with macro-alveoli
spread across the whole volume of the lung

| Average | | | |
|---|---|---|---|
| LE Spacing | Number of | V_{fluid} | ρ_{lung}^{(LE size, LE number)} |
| (mm) | LEs | (mm³) | (g/cm³) |
| — | 0 | 0 | 0.2 |
| 9.5 | 604 | 105791.19 | 0.24 |
| 5.5 | 1495 | 263640.72 | 0.29 |
| 4 | 2284 | 400857.96 | 0.34 |
| 3 | 3173 | 552898.45 | 0.39 |
| 2.3 | 3895 | 678601.29 | 0.44 |

TABLE 2
Lung mass density variation with macro-alveoli
spread across top or bottom half of the lung

| Average | Number of | | |
|---|---|---|---|
| LE | LEs | | |
| Spacing | (top or | V_{fluid} | ρ_{lung}^{(LE size, LE number)} |
| (mm) | bottom half) | (mm³) | (g/cm³) |
| — | 0 | 0 | 0.2 |
| 9.5 | 300 | 52 947.03 | 0.22 |
| 5.5 | 777 | 135 254.91 | 0.25 |
| 4 | 1193 | 200 627.72 | 0.27 |
| 3 | 1583 | 276 638.66 | 0.3 |
| 2.3 | 1930 | 337 930.54 | 0.32 |

### Simulation Platform

A 3D computational human body model with a resolution of 2 mm has been used. The model includes frequency-dependent dielectric properties of 300+ parts of a male human body. These dielectric properties are user-definable in case custom modifications or changes are desired. Although extensive computational time and complexity often create an obstacle in performing sophisticated simulation involving wearables and implants, the use of computational human body models can effectively capture details of the inhomogeneous environment between the antennas. FIG. 14 shows an example of four pairs of transmitter and receiver antennas (two pairs per lung) that are placed on the chest and the back area. For this example, the antenna locations are selected to cover the top and bottom halves of each lung for monitoring purpose. The use of multiple pairs of antennas to cover different areas of both lungs is meant to provide more information about the fluid accumulation in specific area of the lungs.

In general, an array of antenna elements can be assigned to cover each lung from top to bottom. The size of the antennas and the required spacing between them to avoid any cross-coupling would determine the number of antennas that can be used. In-body propagation paths should ensure signal propagation through most of the lung's tissue.

Assuming that N antenna pairs are used to cover each lung, there will be N direct intra-lung propagation channels between the antenna pairs that are directly facing each other at each lung. In addition, there will be N×(N−1) cross intra-lung propagation channels among the antenna pairs placed on one lung, as well as 2N² inter-lung propagation channels between the antennas placed on the left and right lungs. For the example with N=2 shown in FIG. 14, consider the antenna pairs numbered 1 and 2 covering the top and bottom halves of the right lung respectively. Similarly, the antenna pairs numbered 3 and 4 are covering the top and bottom halves of the left lung respectively. Define the propagation channel (i,j) to be the wireless channel between the antenna i on the front and antenna j on the back side of the body covering one of the lungs. Then, we will have two direct intra-lung channels between the antenna pairs 1 and 2 for the right lung (i.e., channels (1, 1) and (2,2)), along with two direct intra-lung channels between the antenna pairs 3 and 4 for the left lung (i.e., channels (3,3) and (4,4)). Also, there are two cross intra-lung channels (1,2) and (2,1) for the right lung plus two cross intra-lung propagation channels (3,4) and (4,3) for the left lung. Likewise, there will be eight inter-lung propagation channels, namely, channels (1,3), (1,4), (2,3), (2,4), (3,1), (3,2), (4,1) and (4,2).

The 3D human body area networking platform at the Information Technology Laboratory of NIST has been used to measure the Forward Transmission coefficient (i.e., S₂₁) of the above channels. Results of observation on correlation among the mass density of the lung and the magnitude or phase of these S₂₁s are provided below.

### Flexible Antenna Design

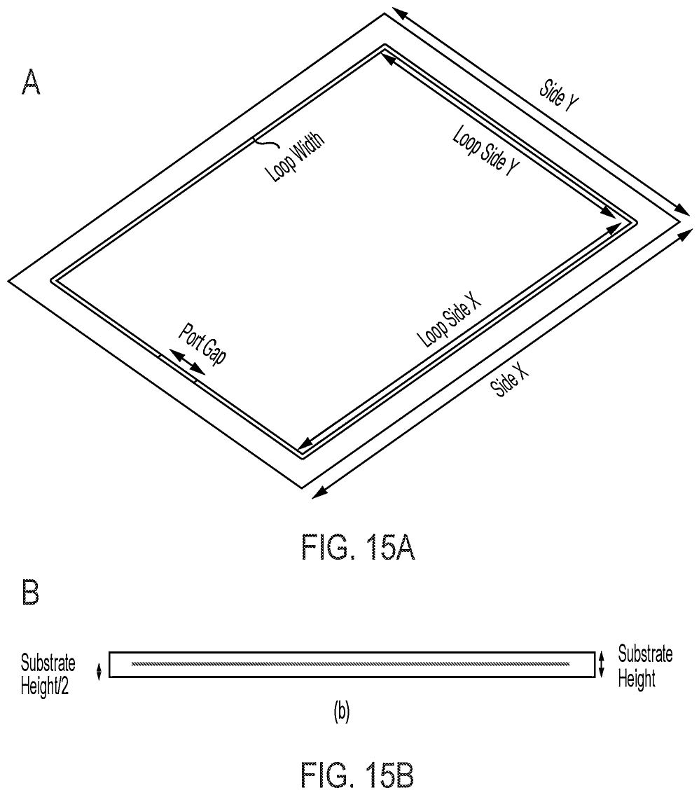

FIG. 15 shows the rectangular loop antenna that has been designed for this application. It operates at the MedRadio band. The loop is sandwiched between two 0.005″ (0.13 mm) thick Rogers 3010 substrates and the overall size is approximately 5 cm by 5 cm. This size is quite acceptable for a wearable antenna specially since the substrate is thin and flexible.

The antenna can be placed directly on the human chest & back areas and is expected to conform to the body surface. Since in practice, the antenna placement varies with different positions and alignment with respect to the lung (considering individuals with different body shapes & sizes), the effective permittivity of the environment exposed to the near field of the antenna could also vary. For the same reason, the optimum dimensions of the antennas that are expected to be placed on the chest and back areas are slightly different. This information is shown in Table 2.

TABLE 3
Dimensions of the flexible loop antenna

| | Transmitter Antenna | Receiver |
|---|---|---|
| Parameters | (mm) | Antenna (mm) |
| Side X | 51.4 | 53.1 |
| Side Y | 43.2 | 41.5 |
| Loop Side X | 49.4 | 47.1 |
| Loop Side Y | 37.2 | 35.5 |
| Loop Width | 0.5 | 0.5 |
| Port Gap | 5 | 5 |
| Substrate Height | 0.26 | 0.26 |

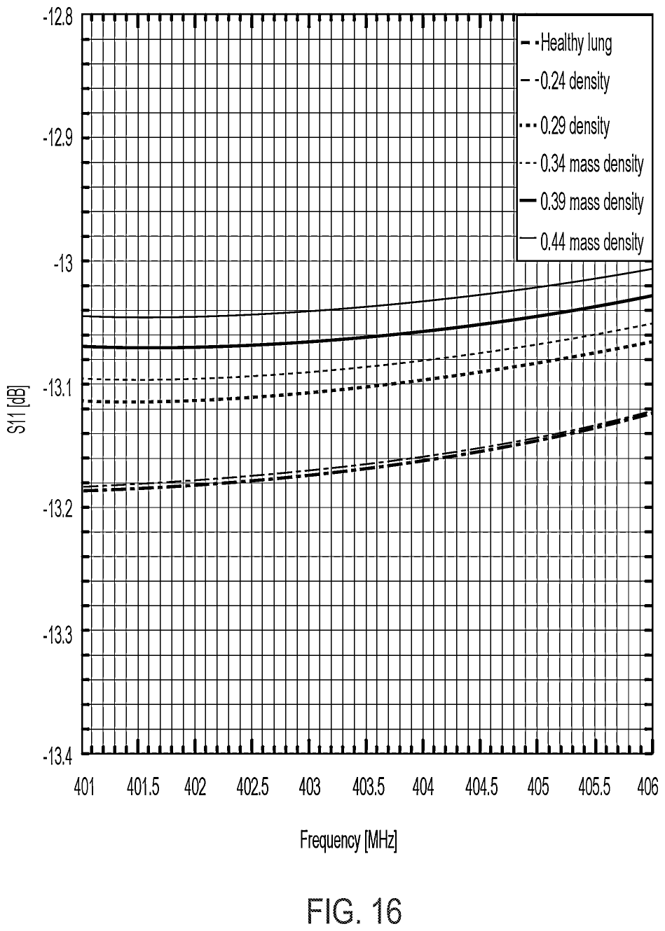

Since the loading effect is unpredictable to some extent, the antenna substrates add a layer of controlled lossless medium in the near field in order to minimize this unpredictable detuning. The return loss displayed in FIG. 16 demonstrates this further. As observed, the matching does not deteriorate with the changing density of the lungs.

### Methodology and Results

Consider that there are N pairs of antennas numbered from 1 to N covering the right lung from top to bottom. Similarly, assume that are also N pairs of antennas covering the left lung (numbered from N+1 on the top to 2N at the bottom). Define Δ_{S}_{₂₁}^{ρ} (i,j) as follows:

$${Δ}_{{S}_{21}}^{ρ} ( i , j ) = ‖ \overline{{S}_{21}} ( i , j ) ‖ - ‖ {S}_{21}^{ρ} ( i , j ) ‖ \qquad ( 1 )$$

where S̅₂̅₁̅(i,j) is the baseline Forward Transmission coefficient of the channel (i,j) when the lung tissue has no excess fluid (i.e., healthy lung); and, S₂₁ρ (i,j) is the Forward Transmission coefficient of the same channel (i,j) when there is excess fluid in the lung resulting in mass density ρ. ∥·∥ denotes the magnitude function.

Also, Δ_{S}_{₂₁}^{ρ} (i,j) represents the deviation in the magnitude of S₂₁ for the channel (i,j) when the mass density of the lung increases to ρ. Now, define the 2N×2N deviation matrix D^{ρ} as follows:

$${D}^{ρ} = {[ {Δ}_{{S}_{21}}^{ρ} ( i , j ) ]}_{2 N \times 2 N} \qquad ( 2 )$$

where i, j=1, 2, 3, . . . , 2N.

For the example in FIG. 12 where N=2, we have:

$${D}^{ρ} = [ \begin{matrix}{Δ}_{{S}_{21}}^{ρ} ( 1 , 1 ) & {Δ}_{{S}_{21}}^{ρ} ( 1 , 2 ) & {Δ}_{{S}_{21}}^{ρ} ( 1 , 3 ) & {Δ}_{{S}_{21}}^{ρ} ( 1 , 4 ) \\ {Δ}_{{S}_{21}}^{ρ} ( 2 , 1 ) & {Δ}_{{S}_{21}}^{ρ} ( 2 , 2 ) & {Δ}_{{S}_{21}}^{ρ} ( 2 , 3 ) & {Δ}_{{S}_{21}}^{ρ} ( 2 , 4 ) \\ {Δ}_{{S}_{21}}^{ρ} ( 3 , 1 ) & {Δ}_{{S}_{21}}^{ρ} ( 3 , 2 ) & {Δ}_{{S}_{21}}^{ρ} ( 3 , 3 ) & {Δ}_{{S}_{21}}^{ρ} ( 3 , 4 ) \\ {Δ}_{{S}_{21}}^{ρ} ( 4 , 1 ) & {Δ}_{{S}_{21}}^{ρ} ( 4 , 2 ) & {Δ}_{{S}_{21}}^{ρ} ( 4 , 3 ) & {Δ}_{{S}_{21}}^{ρ} ( 4 , 4 )\end{matrix} ] \qquad ( 3 )$$

With the specified numbering assignment for the antenna pairs, the 2×2 diagonal submatrices in the matrix D^{ρ} represent the S₂₁ deviation of the direct and cross intra-lung channels for the right and left lungs individually. These submatrices are shown in bold brackets in Equation (4). The 2×2 cross diagonal submatrices shown in parenthesis, on the other hand, represent inter-lung propagation channels between the right and left lungs.

$${D}^{ρ} = [ \begin{matrix}[ \begin{matrix}{Δ}_{{S}_{21}}^{ρ} ( 1 , 1 ) & {Δ}_{{S}_{21}}^{ρ} ( 1 , 2 ) \\ {Δ}_{{S}_{21}}^{ρ} ( 2 , 1 ) & {Δ}_{{S}_{21}}^{ρ} ( 2 , 2 )\end{matrix} ] & ( \begin{matrix}{Δ}_{{S}_{21}}^{ρ} ( 1 , 3 ) & {Δ}_{{S}_{21}}^{ρ} ( 1 , 4 ) \\ {Δ}_{{S}_{21}}^{ρ} ( 2 , 3 ) & {Δ}_{{S}_{21}}^{ρ} ( 2 , 4 )\end{matrix} ) \\ ( \begin{matrix}{Δ}_{{S}_{21}}^{ρ} ( 3 , 1 ) & {Δ}_{{S}_{21}}^{ρ} ( 3 , 2 ) \\ {Δ}_{{S}_{21}}^{ρ} ( 4 , 1 ) & {Δ}_{{S}_{21}}^{ρ} ( 4 , 2 )\end{matrix} ) & [ \begin{matrix}{Δ}_{{S}_{21}}^{ρ} ( 3 , 3 ) & {Δ}_{{S}_{21}}^{ρ} ( 3 , 4 ) \\ {Δ}_{{S}_{21}}^{ρ} ( 4 , 3 ) & {Δ}_{{S}_{21}}^{ρ} ( 4 , 4 )\end{matrix} ]\end{matrix} ] \qquad ( 4 )$$

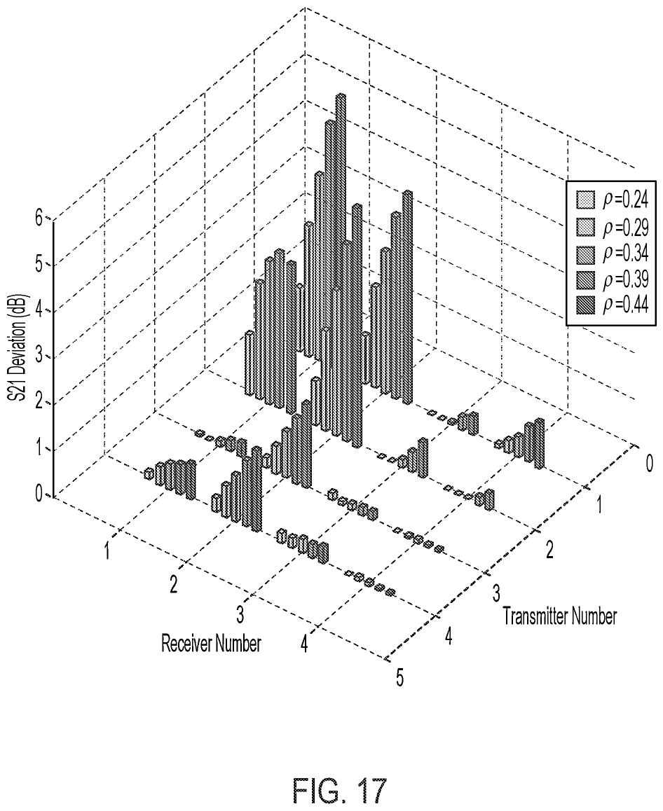

FIG. 17 shows exemplary elements of D^{ρ} when the entire right lung is affected by excess fluid. The numerical values of the S₂₁ deviation (in dB) for each channel (with the lung mass density of ρ) are shown as a bar in this figure. As observed, most of the variations in the S₂₁ magnitude occur in the top left submatrix which represents the right lung. As the mass density of the right lung increases, channels (1,1) and (3,3) experience the biggest variations in the magnitude of their corresponding S₂₁. Radio wave propagation through cross intra-lung channels (1,3) and (3,1) also result in noticeable rise in the S₂₁ deviation. This is because the entire lung tissue is experiencing a change in its effective dielectric properties. FIG. 17. also shows some minor variations in the bottom left submatrix which represents the inter-lung channels with receivers on the back side of the affected (i.e., right) lung. However, the right side of the matrix D^{ρ} does not show any significant variations as the left lung tissue is kept healthy with no excess fluid.

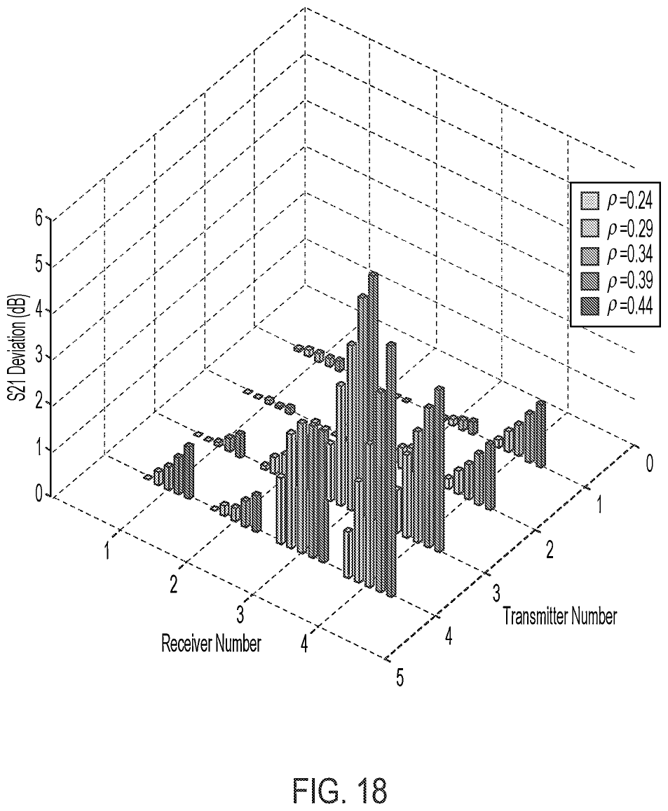

Similar situation exists when only the entire left lung is affected by excess fluid. In that case, the left half of the matrix D^{ρ} does not show any activities and most of the variations will be focused on the lower right submatrix indicating/representing the left lung. This can be seen in FIG. 18.

The variations in the magnitude of S₂₁ versus lung mass density (ρ) can be measured by a wearable device to detect or monitor pulmonary edema or equivalently changes in the amount of fluid in each lung. We propose to use the accumulative effect of relevant deviations in S₂₁ (i.e., elements of matrix D^{ρ}) as a measure of the intensity of pulmonary edema. This will enhance the sensitivity of detection/monitoring through the increased dynamic range in the observed aggregate value of the S₂₁ deviations.

Consider R(ρ) and L(ρ) as indicators of the level of fluid accumulation (or reduction) in the right and left lung respectively. Define R(ρ) and L(ρ) as follows:

$$R ( ρ ) = {\sum }_{j = 1}^{N} {\sum }_{i = 1}^{2 N} {Δ}_{{S}_{21}}^{ρ} ( i , j ) \qquad ( 5 )$$

$$L ( ρ ) = {\sum }_{j = N + 1}^{2 N} {\sum }_{i = 1}^{2 N} {Δ}_{{S}_{21}}^{ρ} ( i , j ) \qquad ( 6 )$$

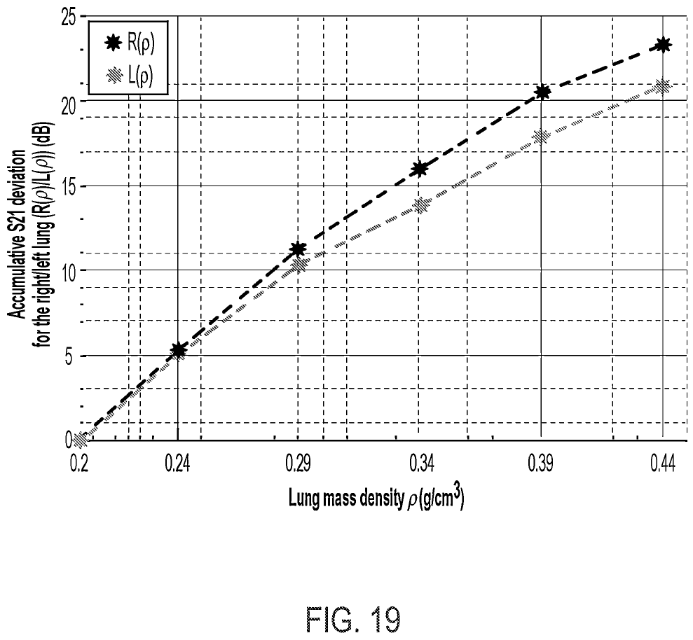

For N=2, FIG. 19 shows the monotonically increasing value of these functions versus p. A zero value for R(ρ)/L(ρ) represents a healthy right/left lung. Compared to a single antenna pair (i.e. N=1), using multiple antennas for each lung increases the monitoring sensitivity by enhancing the capability of detecting smaller changes in the amount of fluid in each lung. FIG. 19 also demonstrates higher aggregate deviation for the right lung (i.e., R(ρ)) compared to L(ρ) as the lung mass density p increases. This is consistent with the fact that the size of the right lung is normally bigger than the left one. As such signal propagation through the bigger volume of the right lung will experience more attenuation compared to the left lung, leading to higher aggregate S₂₁ deviation.

To reduce the required computations or in cases where monitoring a single lung is needed, a smaller D^{ρ} matrix (with dimensions N×N) could be considered to represent a single lung. In that case, the following function M(ρ) can be used as a metric to monitor the accumulation (or reduction) of fluid in the corresponding lung.

$$M ( ρ ) = {\sum }_{j = 1}^{N} {\sum }_{i = 1}^{N} {Δ}_{{S}_{21}}^{ρ} ( i , j ) \qquad ( 7 )$$

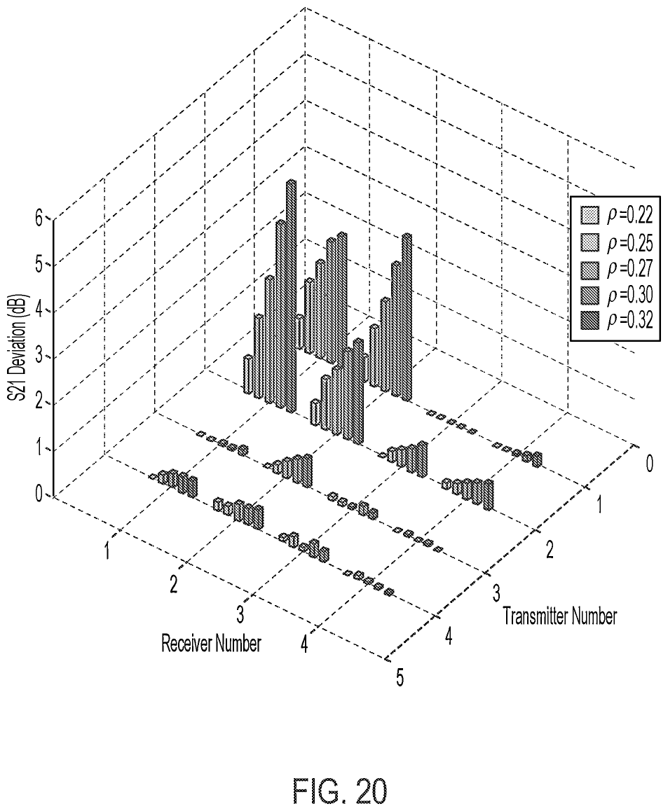

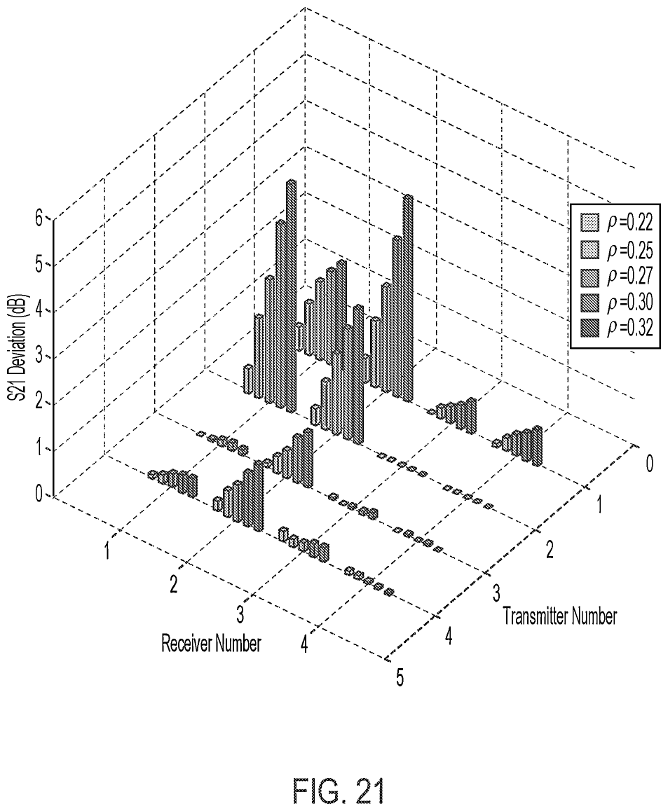

Using multiple pairs of antennas for each lung can also help with the detection of the approximate region where excess fluid could be concentrated. For example, consider the lung model in FIG. 13 (B and C) where half of its volume is affected by excessive fluid. Using the simulation platform, we can obtain matrix D^{ρ} for the case when either top or bottom half of the right lung is experiencing fluid accumulation. FIG. 20 and FIG. 21 show the S₂₁ deviation for all channels in this scenario.

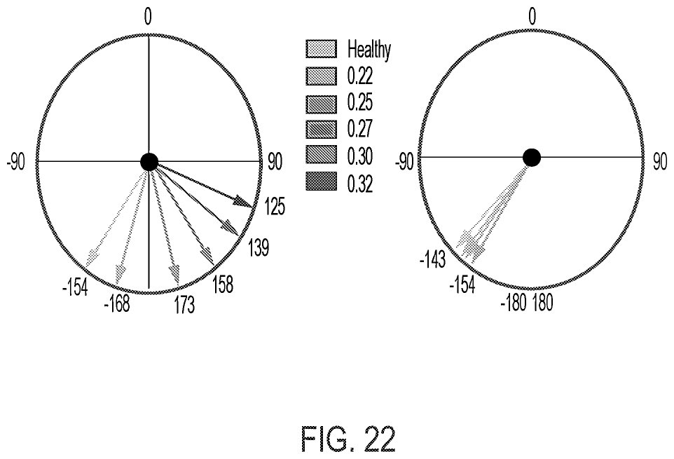

Although, differences in individual channel elements are observed in FIG. 20 and FIG. 21, it is not quite clear how to distinguish between these two cases by merely looking at the magnitudes of S₂₁ deviations or any combinations of the elements of the corresponding matrix D^{ρ}. However, looking at the phase of the forward transmission coefficient of the wireless channels between antenna pairs that are directly facing each other can provide better information about the dielectric content of the channels. For example, lets denote the phase of the S₂₁ for channel (1, 1) when the mass density of the lung is ρ by ∠S₂^{ρ} (1, 1). Comparing the value of this phase when the excess fluid is concentrated in the top versus bottom half of the lung reveals mass density information about these regions. FIG. 22 shows this comparison. With increasing excess fluid in the top half of the right lung, ∠S₂₁^{ρ} (1, 1) monotonically decreases from −154 degrees (for a healthy tissue) to +125 degrees (when ρ=0.32 g/cm³). On the other hand, when the excess fluid is only affecting the bottom half of the lung, changes in the ∠S₂₁^{ρ} (1, 1) are minor and in the opposite direction (i.e., from −154 degrees to −143 degrees). These results indicate a correlation between ∠S₂₁^{ρ} (1, 1) and the mass density (or equivalently the amount of excess fluid) in the top half of the right lung almost independent of the status of the lung tissue in the bottom half.

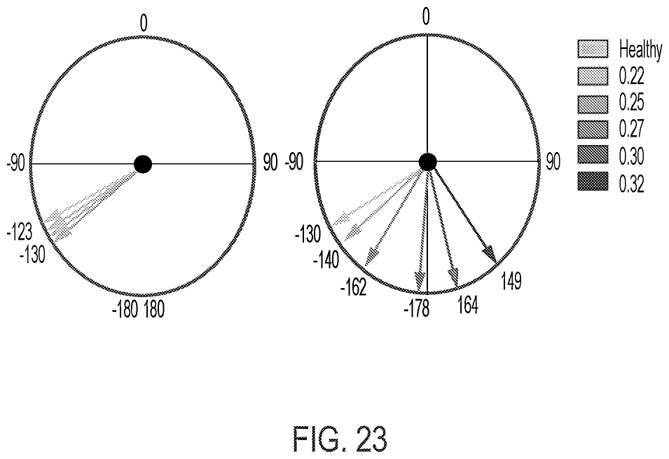

Similar correlation is observed for channel (3,3). As observed in FIG. 23, with increasing excess fluid in the top half of the right lung, ∠₂₁^{ρ} (3,3) exhibits minor changes from −130 to −123 degrees. However, when the excess fluid is affecting the bottom half of the right lung (i.e., the tissue that is directly in between the two antennas), ∠S₂₁^{ρ} (3,3) monotonically decreases from −130 degrees (for a healthy tissue) to +149 degrees (when ρ=0.32 g/cm³).

Similar trend is also observed for ∠S₂₁^{ρ} (2,2) and ∠S₂₁^{ρ} (4,4) when we consider the top versus bottom region of the left lung. There is direct correlation between the ∠S₂₁^{ρ} phase for channels (2,2) and (4,4) and both mass density of the lung tissue in the corresponding region of the lung (i.e., top versus bottom). But most importantly observation of the values of ∠S₂₁^{ρ} (2,2) and ∠S₂₁^{ρ} (4,4) can lead to identifying which region of the lung is affected by excess fluid. These results have been omitted for brevity.

The processes described herein may be embodied in, and fully automated via, software code modules executed by a computing system that includes one or more general purpose computers or processors. The code modules may be stored in any type of non-transitory computer-readable medium or other computer storage device. Some or all the methods may alternatively be embodied in specialized computer hardware. In addition, the components referred to herein may be implemented in hardware, software, firmware, or a combination thereof.

Many other variations than those described herein will be apparent from this disclosure. For example, depending on the embodiment, certain acts, events, or functions of any of the algorithms described herein can be performed in a different sequence, can be added, merged, or left out altogether (e.g., not all described acts or events are necessary for the practice of the algorithms). Moreover, in certain embodiments, acts or events can be performed concurrently, e.g., through multi-threaded processing, interrupt processing, or multiple processors or processor cores or on other parallel architectures, rather than sequentially. In addition, different tasks or processes can be performed by different machines and/or computing systems that can function together.

Any logical blocks, modules, and algorithm elements described or used in connection with the embodiments disclosed herein can be implemented as electronic hardware, computer software, or combinations of both. To clearly illustrate this interchangeability of hardware and software, various illustrative components, blocks, modules, and elements have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks and modules described or used in connection with the embodiments disclosed herein can be implemented or performed by a machine, such as a processing unit or processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, or algorithm described in connection with the embodiments disclosed herein can be embodied directly in hardware, in a software module stored in one or more memory devices and executed by one or more processors, or in a combination of the two. A software module can reside in RAM memory, flash memory, ROM memory, EPROM memory, EEPROM memory, registers, hard disk, a removable disk, a CD-ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The storage medium can be volatile or nonvolatile.

While one or more embodiments have been shown and described, modifications and substitutions may be made thereto without departing from the spirit and scope of the invention. Accordingly, it is to be understood that the present invention has been described by way of illustrations and not limitation. Embodiments herein can be used independently or can be combined.

All ranges disclosed herein are inclusive of the endpoints, and the endpoints are independently combinable with each other. The ranges are continuous and thus contain every value and subset thereof in the range. Unless otherwise stated or contextually inapplicable, all percentages, when expressing a quantity, are weight percentages. The suffix(s) as used herein is intended to include both the singular and the plural of the term that it modifies, thereby including at least one of that term (e.g., the colorant(s) includes at least one colorants). Option, optional, or optionally means that the subsequently described event or circumstance can or cannot occur, and that the description includes instances where the event occurs and instances where it does not. As used herein, combination is inclusive of blends, mixtures, alloys, reaction products, collection of elements, and the like.

As used herein, a combination thereof refers to a combination comprising at least one of the named constituents, components, compounds, or elements, optionally together with one or more of the same class of constituents, components, compounds, or elements.

All references are incorporated herein by reference.

The use of the terms “a,” “an,” and “the” and similar referents in the context of describing the invention (especially in the context of the following claims) are to be construed to cover both the singular and the plural, unless otherwise indicated herein or clearly contradicted by context. It can further be noted that the terms first, second, primary, secondary, and the like herein do not denote any order, quantity, or importance, but rather are used to distinguish one element from another. It will also be understood that, although the terms first, second, etc. are, in some instances, used herein to describe various elements, these elements should not be limited by these terms. For example, a first current could be termed a second current, and, similarly, a second current could be termed a first current, without departing from the scope of the various described embodiments. The first current and the second current are both currents, but they are not the same condition unless explicitly stated as such.

The modifier about used in connection with a quantity is inclusive of the stated value and has the meaning dictated by the context (e.g., it includes the degree of error associated with measurement of the particular quantity). The conjunction or is used to link objects of a list or alternatives and is not disjunctive; rather the elements can be used separately or can be combined together under appropriate circumstances.

What is claimed is:

1. A lung fluid monitor for monitoring fluid level in a lung, the lung fluid monitor comprising:
a radiation source disposable on a first body surface and that produces nascent radiation, the nascent radiation:
being received by the first body surface, communicated from the first body surface to a lung, attenuated proportionately to an amount of fluid in the lung, and communicated from the lung to a second body surface as attenuated radiation;
a radiation detector disposable on the second body surface opposing the first body surface and in electromagnetic communication with the radiation source via the lung and that:
receives the attenuated radiation from the lung and produces a detector signal from the attenuated radiation in response to receiving the attenuated radiation.

2. The lung fluid monitor of claim 1, further comprising an analyzer in electrical communication with the radiation detector and that receives the detector signal from the radiation detector and produces lung health data from the detector signal.

3. The lung fluid monitor of claim 2, wherein the radiation source produces a phase signal that is communicated from the radiation source and received by the analyzer.

4. The lung fluid monitor of claim 3, wherein the radiation source comprises a signal generator that produces a waveform for the nascent radiation.

5. The lung fluid monitor of claim 4, wherein the radiation source further comprises a source antenna that receives the source waveform from the signal generator, produces the nascent radiation from the source waveform, and communicates the nascent radiation to the first body surface.

6. The lung fluid monitor of claim 2, wherein the radiation detector comprises a detection antenna that receives the attenuated radiation from the lung.

7. The lung fluid monitor of claim 6, wherein the radiation detector further comprises data processor that converts the attenuated radiation into the detector signal.

8. The lung fluid monitor of claim 7, wherein the radiation detector further comprises a transmitter that communicates the detector signal to the analyzer.

9. The lung fluid monitor of claim 2, further comprising a housing member on which the radiation source and the radiation detector are disposed.

10. The lung fluid monitor of claim 9, wherein the housing member is wearable garment that is worn by a human.

11. The lung fluid monitor of claim 1, wherein the nascent radiation and the attenuated radiation independently comprise a frequency compliant with the United States of America Federal Communications Communication MedRadio spectrum.

12. The lung fluid monitor of claim 11, wherein the MedRadio spectrum is from 401 MHz to 457 MHz.

13. The lung fluid monitor of claim 1, further comprising additional radiation sources disposed in an array for monitoring the lung.

14. The lung fluid monitor of claim 1, further comprising additional radiation detectors disposed in an array for monitoring the lung.

15. The lung fluid monitor of claim 1, further comprising additional radiation sources disposed in an array; and
additional radiation detectors disposed in an array for monitoring the lung,
wherein the additional radiation sources are in electromagnetic communication with the additional radiation detectors.

16. A process for monitoring fluid level in a lung with a lung fluid monitor, the process comprising:
producing nascent radiation by a radiation source disposed on a first body surface;
communicating the nascent radiation from the radiation source to a lung;
receiving the nascent radiation by the lung;
attenuating the nascent radiation in the lung to produce attenuated radiation from the nascent radiation;
communicating the attenuated radiation from the lung to a radiation detector disposed on a second body surface that opposes the radiation source, such the lung is interposed between the first body surface and the second body surface;
receiving, by the radiation detector, the attenuated radiation from the lung;
producing, by the radiation detector, a detector signal from the attenuated radiation;
communicating the detector signal from the radiation detector to a analyzer;
receiving, by the analyzer, the detector signal from the radiation detector;
analyzing, by the analyzer, the detector signal;
producing, by the analyzer, lung health data from analyzing the detector signal to monitor the fluid level in a lung.

17. The process of claim 16, further comprising communicating a phase signal from the radiation source to the analyzer.

18. The process of claim 16, further comprising determining a lung region of the lung corresponding to the lung health data.

19. The process of claim 16, wherein the lung health data comprises a regional fluid accumulation in a lung region

20. The process of claim 16, wherein the lung health data comprises a temporal history a change in an amount of fluid in the lung.
